(19) United States
(12) Patent Application Publication
(10) Pub. No.: US 2003/0133829 A1
(43) Pub. Date: Jul. 17, 2003

(54) Process for inactivating pathogens in a biological material

(75) Inventors: Heinz Anderle, Klosterneuburg (AT); Martin Spruth, Vienna (AT); Peter Matthiessen, Vienna (AT); Peter Turecek, Klosterneuburg (AT); Thomas Kreil, Klosterneuburg (AT); Hans-Peter Schwarz, Vienna (AT)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Baxter Healthcare Corporation, Irvine, CA

(21) Appl. No.: 10/029,501

(22) Filed: Dec. 21, 2001

(51) Int. Cl. A61L 2/00; C12N 1/06

(52) U.S. Cl. 422/28; 435/259

(57) ABSTRACT

The present invention provides a method for inactivating pathogens in a protein solution. The method includes adding to the protein solution either separately or together as a composition (a) a detergent; and (b) an ester of a di- or tri-carboxylic acid to make a preparation. The ester is generally present in the preparation in a concentration of from about 0.001 to about 2% (w/w). The detergent is typically present in a concentration of about 0.001 to about 2% (w/w). The preparation is incubated for a sufficient amount of time sufficient to inactivate the pathogens.

### FIELD OF THE INVENTION

The present invention relates to a method for inactivating pathogens in a protein solution and a method for increasing the pathogen inactivating properties of a composition.

### BACKGROUND OF THE INVENTION

The inactivation of lipid-enveloped viruses by treatment with a detergent alone, or in combination with a solvent to disrupt the outer lipid envelope is a well established viral inactivation method and used in manufacturing human plasma-derived biological products.

One of the first methods of using detergents, which claimed to be successful in the inactivation of Hepatitis B virus (HBV) dates back to 1980 (U.S. Pat. No. 4,314,997). Another method (U.S. Pat. No. 4,370,264) disclosed a three-step hepatitis B inactivation with Tween 80 addition, beta-propiolactone addition, and ultraviolet irradiation, and a final adsorption of the hepatitis B antigen on fumed silica. The emergence of HIV and the outbreak of AIDS in the early 1980s in recipients of plasma-derived coagulation factor concentrates necessitated the need to develop effective viral inactivation methods which were based mainly on dry, vapor, and wet heat treatments. The use of solvents, such as diethyl ether or alcohols, with a detergent at low temperatures (4° C.) was attempted, but did not prove to be practical (U.S. Pat. No. 4,481,189). In 1985, the use of the solvent extractant, tri-n-butylphosphate (TNBP) with Na-deoxycholate for the inactivation of HIV and HBV was described (U.S. Pat. No. 4,540,573; 4,764,369; and 4,820,805); today the standard combinations of polysorbates (Tween) (Brandner J D, Drug Dev. Ind. Pharmacy 24(11): 1049-1054 (1998)), or Tween and Triton X-100 with TNBP are widely applied for plasma-derived products, such as FVIII, and IgG. FVIII activity is retained. The solvent/detergent (S/D) treatment with non-ionic solvents is compatible with large-scale column chromatography on ion-exchange resins, and is used for treating pooled plasma with subsequent back-extraction of the reagents and removal by hydrophobic chromatography.

Other methods based on the use of lipophilic substances for the inactivation of lipid-enveloped viruses include treatment with oleic acid, its esters or alkali or alkaline earth salts (U.S. Pat. No. 4,613,501); the addition of monounsaturated fatty acids with a carbon chain length of 16 C atoms or more, their esters or alkali or alkaline earth salts, their corresponding fatty alcohols, or their fatty acid monoacyl glycerides (U.S. Pat. No. 4,841,023), the addition of caprylic acid in a pH range from 4 to 8 (U.S. Pat. No. 4,939,176), and the addition of a tenside to a concentration equal to or greater than 1% by weight (U.S. Pat. No. 5,639,730; and U.S. Pat. No. 5,733,885).

There is, however, a serine protease group, which includes the prothrombin complex of coagulation factors and some other blood proteins, such as alpha 1-antitrypsin, that are sensitive to and at least partially deactivated by the conventional S/D method. For these proteins, a detergent alone may be used at high concentrations. The most commonly used detergent, Tween 80 (sorbitan-polyoxyethylene-monooleate), increases the viscosity of the protein solution, rendering the undiluted protein/detergent mixture less suitable for column chromatography. Diluting the protein detergent mixture to a greater volume leads to an increase in the time to load the column.

The addition of a low concentration of Tween 80 (˜1%) to a plasma protein solution is ineffective against HBV (U.S. Pat. No. 4,370,264. ), as demonstrated by a reduction of the hepatitis B surface antigen concentration from 2700 to 170 ng/mL, and a reduction of the lipid-enveloped coliphage T2 from 20000 to 10000 viruses/mL at a concentration of 3% Tween 80. If this detergent is used alone, the double-stranded, lipid-enveloped DNA virus pseudo-rabies virus (PRV), a herpes virus, is the most resistant of the three viruses required for validation of a lipid-enveloped virus inactivation method; human immunodeficiency virus (HIV) and bovine viral diarrhoea virus (BVDV) are models for Hepatitis C and are more sensitive towards Tween 80. When used alone, Tween 80 must be used in higher concentrations (5-15% w/v) to effectively inactivate the PRV model virus for Hepatitis B.

Tween 80, originally prepared from bovine tallow, has been replaced with vegetable-derived Tween 80, which is processed from corn oil. The higher content of oligo-unsaturated fatty acids (e.g. linoleic acid) in corn oil, compared to bovine tallow, is presumably reduced by partial hydrogenation. Residual linoleic acid, a di-unsaturated acid, in corn oil-derived Tween 80 has been postulated to form peroxides, which can damage proteins (Roberts et al., Biologicals 27(3): 263-264 (1999)). Therefore, the use of a lower concentration of this detergent is desirable.

Citric acid esters are known plasticizers in food packaging materials and in pharmaceutical coatings. The esters have also been used as carriers for dissolved artificial and natural flavors. Citric acid esters are relatively non-toxic and are rapidly metabolized (Finkelstein et al., Toxicol. Applied Pharmacol. 1: 283-298 (1959)). Citric acid esters have also been described as auxiliary diluents and solvents for virucidal compounds, but have not been recognized as capable of inactivating pathogens in a protein solution. The structurally related glycerol-fatty acid esters triacetin and tributyrin are also generally regarded as being safe and of minimal toxicity.

U.S. Pat. No. 5,492,692 discloses a composition having anti-HIV activity. The composition includes specific oligomers, with polyvinylpyrrolidone-iodine complexes. Furthermore, it is disclosed that surfactants may be incorporated into the mixture. Also disclosed is the use of triethylcitrate or triacetin as a plasticizer in a concentration of between 2.8 and 5%. The claimed antiviral activity is, however, due to the specific oligomers and the polyvinylpyrrolidone-iodine complexes.

U.S. Pat. No. 5,824,708 discloses virucidal cleaning preparations, which include an active antiviral ingredient. The active ingredient is a reaction product between an N-substituted propylenediamine and an ester of glutamic acid. The active ingredient is present in the virucidal preparation in an amount of about 0.005% by weight to about 3% per weight. Furthermore, the preparation includes solvents such as triethylacetate and triethylcitrate in quantities of about 3 to 40% by weight based on the total formulation. The solvents are present in the preparation “in addition to the active ingredient,” i.e., the glutamic acid reaction product. Additionally, a non-ionic surfactant may be added in quantities of about 3 to 20% by weight. The '708 patent does not, however, suggest that the aqueous carrier and solvent are effective antiviral agents in the absence of the active ingredient.

U.S. Pat. No. 6,165,493 is directed to a method for decreasing the frequency of transmission of human cytomegalovirus. The '493 patent discloses that it was known in the art to combine hydroxypropyl methylcellulose acetate (HPMCP) with a plasticizer, such as triacetin in an oral formulation to prevent the HPMCP from cracking.

In view of the deficiencies of prior art pathogen inactivation methods, a gentle, effective procedure for inactivating pathogens in a protein solution, which does not substantially reduce the activity of a selected protein in the solution, would represent a significant advance in the art. The present invention provides such a method.

### BRIEF SUMMARY OF THE INVENTION

The inventors have discovered that a solvent/detergent reagent mixture, which is highly effective for inactivating pathogens, but which has no denaturing effect on selected blood proteins can be prepared from a carboxylic acid ester combined with a surfactant. Furthermore, the use of advanced purification methods, such as column chromatography, is facilitated by the use of a composition that has a viscosity lower than that of the presently used Tween solutions.

In a first aspect, the invention provides a method for inactivating pathogens in a protein solution. The method includes adding to the protein solution, either separately or in combination (a) from about 0.01 to about 2% by weight based on the total weight of the composition of a detergent; and (b) from about 0.001 to about 2% by weight based on the total weight of the composition of a carboxylic acid ester. The resulting mixture is then incubated for an amount of time sufficient to inactivate the pathogens. With this specific combination of a carboxylic acid ester and detergent it has been surprisingly found that pathogens in a protein solution are effectively inactivated, while the protein activity is substantially fully preserved.

In a second aspect, the invention provides a method for increasing the pathogen inactivation properties of a detergent-containing composition. The method includes adding to the detergent-containing composition an amount of a carboxylic acid ester sufficient to increase the pathogen inactivating properties of the composition.

Other aspects, advantages and objects of the present invention will be apparent from the detailed description that follows.

### DETAILED DESCRIPTION OF THE INVENTION

Definitions

As used herein, the term “pathogens” refers to all types of microorganisms, including viruses, which can cause illness and weakness in humans and animals.

As used herein, the phrase “to inactivate a pathogen” refers to significantly reducing the pathogenic properties of a microorganism. Significant reduction generally refers to a reduction in pathogenicity of at least about 50%, at least about 70%, at least about 85%, at least about 95%, or at least about 99% relative to the pathogenicity before the inactivation step.

As used herein, the term “inactivating” relates to eliminating the pathogenic properties of all or a specific family of pathogens present so that any danger of transmitting pathogens or causing an illness or weakness through the protein solution is totally eliminated.

Methods of detecting pathogen inactivation are generally known to those of skill in the art. In an exemplary method, inactivation is demonstrated by adding a small volume fraction (1/10-1/1000 of the protein solution) of a high-titer virus stock solution (˜18 tissue-culture infectious doses TCID₅₀ for mammalian cell viruses or plaque-forming units for bacteriophages) obtained from a virus-infected cell culture supernatant or a bacteriophage-infected host-bacteria lysate, to the sample solution, subjecting the virus-spiked protein solution to the virus inactivation treatment, and drawing samples before, during, and after the time necessary to inactivate a selected amount of virus. The samples are then diluted serially (in decadic logarithmic or half-logarithmic dilutions) and used for the infection of host cells, such as cultured mammalian cells or bacteria suspensions. The titer is determined from the number of virus-infected tissue cultures displaying a cytopathic effect, or the number of bacteriophage plaques in the bacteria layer plated on a single petri dish, at a defined dilution.).

The terms, “substantially intact,” “substantially non-deactivated” and “substantially undenatured,” refer to the activity of a protein in a solution that has been treated by a method of the invention. The terms refer to protein activity, which is at least about 70%, at least about 80%, at least about 90%, at least about 95%, or at least about 99% of that of a protein that has not been submitted to treatment by a method of the invention. Standard assays for determining protein activity are known to those of skill in the art and the selection of an appropriate assay for a particular protein is well within the abilities of one of skill.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below as “heteroalkyl.” Alkyl groups, which are limited to hydrocarbon groups are termed “homoalkyl”.

The term “alkylene” by itself or as part of another substituent means a divalent radical derived from an alkane, as exemplified, but not limited, by —CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃—CH_{2—S—CH}₂—CH₃, —CH₂—CH₂, —S(O)—CH₃, —CH₂—CH₂—S(O)₂—CH₃, —CH═CH—O—CH_{3 , —Si(CH}₃)₃, —CH₂—CH═N—OCH₃, and —CH═CH—(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified by —CH₂CH₂—S—CH₂CH₂- and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied.

The term “aryl” means, unless otherwise stated, a polyunsaturated aromatic, hydrocarbon substituent, which can be a single ring or multiple rings (up to three rings), which are fused together or linked covalently. The term “heteroaryl” refers to aryl groups (or rings) that contain from zero to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) include both substituted and unsubstituted forms of the indicated radical. Preferred substituents for each type of radical are provided below.

Each of the above terms (e.g., “alkyl,” “heteroalkyl,” “aryl” and “heteroaryl”) is meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, -NRSO₂R′, —CN and —NO₂ in a number ranging from zero to (2 m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄) alkyl, in a number ranging from zero the total number of open valences on the aromatic ring system; and where R′, R″, R′″ and R″″are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

As used herein, the term “heteroatom” is meant to include oxygen (O), nitrogen (N), sulfur (S) and silicon (Si).

As used herein, the term “preparation” is meant to comprise the protein or protein solution to be treated in combination with the carboxylic acid ester and detergent.The Method

As discussed in the preceding paragraphs, the present method provides a method for inactivating a pathogen in a protein solution, while leaving the activity of the protein substantially intact.

Thus, in a first aspect, the invention provides a method for inactivating pathogens in a protein solution. The method includes adding to the protein solution, either separately or in combination a detergent and a carboxylic acid ester to form a preparation. In an exemplary embodiment, the detergent is present in the preparation in a concentration from about 0.01 to about 2% (w/w) of a detergent, and the carboxylic acid ester is present in a concentration of from about 0.001 to about 2% . With this specific combination of a carboxylic acid ester and detergent it has been found that pathogens in a protein solution are effectively inactivated, while the protein activity remains substantially intact.

The present invention provides a method for quickly deactivating a pathogen in a protein mixture. The carboxylic acid ester/detergent composition of the invention and the protein solution are incubated for an amount of time sufficient to inactivate at least one pathogen present in the protein solution. In an exemplary embodiment, the mixture is incubated for at least about 0.5 min. There is no upper time limit for the incubation period. In certain embodiments, the use of the method of the invention results in a reduction in the incubation time necessary to inactivate the pathogen, relative to the incubation time necessary for a composition that includes the detergent or the ester alone.

The mixture can be incubated at ambient temperature. Alternatively, the mixture can be heated or cooled as appropriate. In an exemplary embodiment, the mixture is incubated at a temperature from about 0° C. to about 60° C. depending on the combination of the ester and the detergent and the pathogens present in the protein solution. In another representative embodiment, the mixture is incubated at a temperature of from about 10° C. to about 40° C.

In an exemplary embodiment, the method of the invention provides for significant inactivation of a pathogen after an incubation of about 60 minutes. For example, the addition of 0.15% tributyl citrate (w/w) and 1% Tween 80 (w/w) to FEIBA-eluate spiked with PRV reduces the TCID₅₀ from 7.13 to <1.61 after 60 min at 40° C., as demonstrated in Example 5.

In exemplary embodiments, the protein treated by a method of the invention retains more activity than the same protein treated with the art-recognized S/D treatment. Generally preferred are treatment conditions that result in a protein with from about 5% to about 50% more activity than the same protein submitted to S/D treatment. For example when the protein is FIX or FX, and the ester is acetyl triethyl citrate the protein retains from at least about 10% to at least about 25% more activity than the same protein treated with the art-recognized S/D treatment.

In a second aspect, the invention provides a method for increasing the pathogen inactivation properties of a detergent-containing composition. The method includes adding to the detergent-containing composition an amount of a carboxylic acid ester sufficient to increase the pathogen inactivating properties of the composition.

The Preparation

As discussed above, the present invention provides a method of inactivating pathogens, which utilizes a preparation that includes a carboxylic acid ester and a detergent. The preparation is typically an aqueous formulation. The preparation can include other species in addition to the ester and the detergent. For example the preparation can include organic co-solvents, buffers, salts, preservatives, and the like.

The preparation of the invention does not generally include free cc-amino acids, or a-amino acid amides, such as glutamic acid and amides of glutamic acid, particularly those glutamic acid derivatives that are described in U.S. Pat. No. 5,824,708.

The Carboxylic Acid Ester

Esters useful in practicing the present invention are formed between carboxylic acids and alcohols of substantially any structure. For example, mono-, di- and tri-carboxylic acids are of use in the methods described herein. The carboxylic acids can include diverse structures within their framework, e.g., substituted or unsubstituted alkyl, substituted or unsubstituted heteroalkyl and substituted or unsubstituted aryl groups.

Exemplary classes of carboxylic acids of use in the invention include one or more meta- or ortho-carbonic acid, a monocarboxylic acid, an oligocarboxylic acid, a hydroxy mono- or oligo-carboxylic acid, and a keto-mono carboxylic acid or -oligocarboxylic acid. Exemplary members of the above-recited classes include acetic acid, butyric acid, adipic acid, sebacic acid, succinic acid, fumaric acid, lactic acid, glycolic acid, malic acid, tartaric acid, mono- and di-acetyl tartaric acid, citric acid, isocitric acid, gluconic acid, pyruvic acid and oxaloacetic acid.

In an exemplary embodiment, the ester is derived from a short chain substituted or unsubstituted alkyl or heteroalkyl carboxylic acid, such as C₁-C₁₄ acids, C₂-C₁₂ acids, C₃-C₀₁ acids, or C₄-C₈ acids. The ester typically will not be formed between an alcohol and oleic acid or an adjacent homolog thereof. Thus, another exemplary subset of mono-carboxylic acids of use in the invention includes C₁-C₁₅ and C₂₀ and longer saturated acids.

The carboxylic acid is esterified with an alcohol. The alcohol component of the carboxylic acid esters of use in the present invention can be derived from substantially any alcohol. Alcohol components of the esters of use in the present invention include, but are not limited to, mono-hydroxy alcohols, di-hydroxy alcohols, tri-hydroxy alcohols and poly-hydroxy alcohols. Exemplary alcohols include within their framework one or more groups such as substituted or unsubstituted alkyl, substituted or unsubstituted heteroalkyl and substituted or unsubstituted aryl.

Exemplary alcohols include methanol ethanol, n-butanol, dodecanol, tetradecanol, hexadecanol, octadecanol, eicosanol, glycerol, threitol, erythritol, pentitols and hexitols, pentose or hexose monosaccharides, and pentose or hexose oligosaccharides. Presently preferred alcohols are short chain alcohols, which have a positive effect on the solubility of the ester in the preparation, such as substituted or unsubstituted C₁-C₄ alkyl alcohols.

The ester will generally not be an ester of oleic acid or an adjacent homolog. When an ester of oleic acid or an adjacent homolog is used, the ester will typically not be an ester formed between a C₁-C₄ alcohol (e.g., methanol, ethanol) and oleic acid or the adjacent homolog.

The concentration of the ester in the preparation is generally between about 0.01 and about 1% (w/w), between about 0.05 and about 0.5% (w/w), or between about 0.06 and about 0.36% (w/w). According to the present invention, the presence of the ester in the composition, even at low concentrations enhances the effectiveness of the detergent. In particular, the combination of a carboxylic acid ester and a Tween 80 is extremely effective for the inactivation of pathogens.

The Detergent

Substantially any detergent, whether anionic, cationic or neutral can be used to practice the present invention. Exemplary detergents include alkali metal salts of fatty acids, cholic (bile) acids, sodium- or calcium-stearoyl lactyl 2-lactate, a short-chain (<C₁₄) fatty acid mono- or di-glyceride), a fatty acid mono- or di-glyceride which is esterified with acetic acid; lactic acid; citric acid; tartaric acid; mono- or di-acetyl tartaric acid; sugar-fatty acid esters; sugar glycerides; sorbitan-fatty acid esters; sorbitan-polyoxyethylene-fatty acid esters (polysorbates); and octoxynol 9 (triton X-100) and nonoxynol 9. Also of use are glycerides having two or more different residues derived from different carboxylic acids, e.g, acetic acid and tartaric acid. Selection one or more appropriate detergent for a particular application is within the abilities of one of skill without the need for undue experimentation.

An exemplary Tween detergent of use in the invention is Tween 80. Tween 80 is a known detergent, which has been used for the inactivation of pathogens in a biological sample.

The amount of detergent is generally between about 0.1 and about 3% (w/w), e.g., about 1% (w/w). Even at low concentrations the detergent, in particular Tween 80, effectively inactivates pathogens such as HIV and its model virus PRV, a result not obtained with the detergent alone.

The Protein

The method of the invention can be used to deactivate pathogens in a preparation of substantially any protein or a protein-containing solution, such as plasma or an immunoglobulin solution. Exemplary proteins include coagulation factors, immunoglobulins, albumin, antithrombin III, alpha-1-antitrypsin, C1-esterase inhibitor and FEIBA. Such coagulation factors include FII, FV, FVII, FVIII, FIX, FX, FXI, FXII, FXIII. The proteins can be either purified from a natural source, e.g. plasma, or recombinantly or transgenically produced. The above-recited proteins are sensitive to conventional S/D methods, however, with the method according to the present invention these proteins remain stable and fully active even after complete inactivation of any pathogen present in the protein solution.

According to an advantageous embodiment of the present invention the biological reactive protein is enriched from a natural source by chromatographic or precipitation methods. Methods of protein enrichment are well known to those of skill in the art. Moreover, the choice of an efficacious method for enriching a particular protein is well within the abilities of those of ordinary skill. In addition to a protein enrichments step, the protein may also be purified either before or after the treatment disclosed herein.

The protein is isolated from substantially any source. It can be natural, or recombinantly derived. Natural sources are, for example, body fluids, in particular blood or plasma, as well as body tissue and the like.

According to another embodiment of the present invention the protein is a recombinantly produced protein or a transgenically produced protein. In one embodiment, the recombinantly produced protein is isolated from a selected transgenic plant or animal. In another embodiment, the protein is produced in cell culture. The cells are stably or transiently transfected with the specific gene. Such recombinant and transgenic methods are well known to the person skilled in the art and it is well within the abilities of one of skill to select and advantageous method to prepare and/or isolate the selected protein. When the protein preparation includes more than one protein, the different proteins are optionally purified from a natural, recombinant or transgenic source.

The Pathogens

The method of the present invention is useful to inactivate substantially any pathogen. Exemplary pathogens include lipid-enveloped viruses, such as hepatitis B virus, hepatitis C virus, hepatitis D (delta) virus, hepatitis G virus, human immunodeficiency virus, bovine viral diarrhoea virus and herpes virus pseudorabies virus (PRV). Even PRV, which is the most resistant of the viruses required for validation of a lipid-enveloped virus inactivation method is completely inactivated with the method according to the present invention.

A further aspect of the present invention relates to a method for enhancing the pathogen inactivating properties of a composition comprising at least one detergent. The method includes adding to the composition an ester of a carboxylic acid and an alcohol. For this method the above mentioned definitions and preferred embodiments also apply.

The materials, and methods of the present invention are further illustrated by the examples, which follow. These examples are offered to illustrate, but not to limit the claimed invention.

### EXAMPLES

Materials and Methods

The so-called “partial prothrombin complex” (PPC) of the coagulation factors II, IX, X and protein C is isolated from the cryoprecipitation supernatant by adsorption on DEAE-sephadex anion exchange resin. It is then eluted and concentrated to obtain either the PPC component of the total prothrombin complex (with FVII) or an activated prothrombin complex concentrate, the “Factor Eight Inhibitor Bypassing Activity” (FEIBA). Both products are virally inactivated in a single step by vapour heat treatment. A second independent step for lipid-enveloped viruses is required for the next product generation. The DEAE sephadex eluate can be incubated with a detergent, then diluted and the coagulation factors readsorbed on DEAE sephadex gel. The addition of a high concentration of Tween 80 requires extensive washing of the protein-loaded gel in five repeated steps to remove the detergent.

To PPC DEAE sephadex eluate 1% (w/w) Tween 80 and 0.36% (w/w) acetyl triethyl citrate (ATEC) were added and the solution was incubated for 200 min at 40° C. A parallel run was done with S/D (1% Tween 80 and 0.3% TNBP). During the first 30 min of Tween/ATEC treatment, the emulsion clarified to a homogeneous solution, while the S/D emulsion remained turbid. FIX activity was reduced about 10% with Tween/ATEC and about 30% with S/D, while FX activity was retained with Tween/ATEC but reduced about 20% with S/D.

To FEIBA DEAE sephadex eluate 1% (w/w) Tween 80 and 0.3% (w/w) ATEC were added and the solution incubated for 180 min at 40° C. After five-fold dilution with water, the proteins were readsorbed on DEAE sephadex gel. The protein-loaded gel was suction-filtered, re-suspended and washed four times to remove the detergent. The proteins were eluted and concentrated by ultra-diafiltration, and FEIBA was generated by incubation. The FEIBA generation rate was equivalent to the FEIBA VH product.

### EXAMPLE 1

Cohn-IV/1 paste (Cohn EJ et al., J. Am. Chem. Soc. 68 (1946), 459-475) was dissolved in a 24-fold volume of 30 mM Tris buffer, pH=6.4, and sterile-filtered through a 0.22 μm membrane filter. Incubation of the obtained alpha 1-antitrypsin purification starting material was done at 40° C. with various combinations of trialkyl citrate or triacyl glyceride; the elastase-inhibiting activity (EIA) of alpha 1-antitrypsin is given in % untreated.

TABLE 1
(All percents are (w/w)

| | | 0.1% | | | 0.5% | 0.5% | | 0.25% | |
|---|---|---|---|---|---|---|---|---|---|
| time | 0.1% | tributyl | 0.5% tri- | 0.5% | triethyl/ | tributyl | 0.25% | tributyl | 0.3% TNBP/ |
| (min) | tributyrin/ | citrate | butyrin/ | triacetin/ | citrate | citrate/ | tributyrin | citrate/ | 1% Tween |
| at | 1% Tween | 1% Tween | 1.5% Tween | 1.5% Teen | 1.5% Tween | 1.5% Tween | 1% Tween | 1% Tween | (S/D) |
| 40° C. | EIA | EIA | EIA | EIA | EIA | EIA | EIA | EIA | EIA |
| untr. | 100.00% | 100.00% | 100.00% | 100.00% | 100.00% | 100.00% | 100.00% | 100.00% | 100.00% |
| 30 | 120.08% | 117.23% | 151.11% | 114.25% | 118.30% | 115.42% | 127.54% | 113.98% | 120.33% |
| 60 | 122.93% | 121.03% | 151.11% | 114.25% | 114.38% | 116.34% | 129.58% | 109.91% | 84.10% |
| 90 | 107.73% | 114.38% | 125.62% | 123.07% | 126.14% | 120.26% | 108.82% | 111.94% | 112.20% |
| 120 | 89.82% | 127.68% | 142.61% | 95.79% | 126.14% | 111.37% | 108.82% | 110.85% | 71.90% |
| 180 | 92.67% | 123.88% | 90.20% | 106.92% | 113.33% | 113.33% | 95.25% | 115.06% | 95.93% |

As shown in Table 1, the elastase-inhibiting activity remained most stable through the treatment with tributyl citrate and Tween 80 in combination.

### EXAMPLE 2

Partial prothrombin complex eluate (PPC) was obtained from cryoprecipitation supernatant by the adsorption on DEAE-Sephadex A 50 anion exchange gel and subsequent concentrated elution (Brummelhuis HGJ: Preparation of the prothromin complex, in: Curling JM: Methods of plasma protein fractionation, London: Academic Press 1980, 117-128). Incubation this of partial prothrombin complex (PPC) DEAE eluate was done at 40° C. with 1% Tween 80+0.15% tributyl citrate (1,15) or 2,5% Tween 80 and 0.375% tributyl citrate (2,875); coagulation factor activity measurement was done by coagulation time measurement for prothrombin (FII) and FIX, by the chromogenic assay for FX, and by the amidolysis assay for protein C (PC).

TABLE 2

| time (min) | | | | | | | | |
|---|---|---|---|---|---|---|---|---|
| at 40° C. | % FII 1,15 | % FII 2,875 | % FIX 1,15 | % FIX 2,875 | % FX 1,15 | % FX 2,875 | % PC 1,15 | % PC 2,875 |
| untreated | 100.0% | 100.0% | 100.0% | 100.0% | 100.0% | 100.0% | 100.0% | 100.0% |
| 10 | 138.8% | 139.7% | 145.1% | 124.9% | 105.0% | 100.4% | 117.1% | 121.6% |
| 60 | 140.0% | 133.4% | 109.7% | 139.1% | 104.3% | 101.9% | 117.4% | 120.2% |
| 120 | 132.9% | 138.1% | 106.8% | 119.2% | 105.3% | 99.9% | 118.0% | 116.7% |
| 180 | 133.0% | 149.5% | 135.9% | 110.2% | 104.2% | 97.1% | 116.6% | 116.9% |
| 240 | 142.5% | 139.4% | 139.4% | 126.3% | 101.8% | 99.1% | 117.4% | 121.8% |

As shown in Table 2, the treatment with tributyl citrate and Tween 80 did not reduce the coagulation factor activity.

### EXAMPLE 3

PPC DEAE eluate (as described in Example 2) was incubated with: (1) 0.36% acetyl triethyl citrate; or (2) 1% Tween 80 (A/T) and 0.3% tributyl phosphate; or (3) 1% Tween 80 (S/D) at 40° C. The coagulation factor activity was determined using the same methods as in Example 2.

TABLE 3

| time (min) | [%] FII | [%] FII | [%] FIX | [%] FIX | [%] FX | [%] FX | [%] PC | [%] PC |
|---|---|---|---|---|---|---|---|---|
| at 40° C. | A/T | S/D | A/T | S/D | A/T | S/D | A/T | S/D |
| PPC untr. | 100% | 100% | 100% | 100% | 100% | 100% | 100% | 100% |
| 35 | 103.5% | 116.6% | 97.3% | 80.2% | 103.3% | 97.2% | 101.2% | 105.4% |
| 75 | 128.6% | 106.9% | 95.7% | 90.8% | 102.5% | 90.5% | 99.2% | 107.5% |
| 130 | 122.4% | 108.9% | 114.2% | 82.4% | 102.8% | 85.9% | 97.3% | 106.0% |
| 200 | 121.2% | 114.7% | 92.5% | 72.4% | 101.6% | 78.2% | 93.1% | 105.0% |

As shown in Table 3, the coagulation factor FX was more highly denatured by S/D treatment than by treatment with tributyl citrate/Tween 80 or acetyl triethyl Tween 80.

### EXAMPLE 4

Partial prothrombin complex DEAE eluate (see, Example 2) was incubated with 1% Tween 80 and 0.36% acetyl triethyl citrate for 180 min at 40° C., with subsequent 5-fold dilution with water, re-adsorption on DEAE sephadex A50 gel (12 g/L initial volume of PPC DEAE eluate), 4 repeated washing steps with the initial PPC DEAE eluate volume of 0.2 M NaCl+0.0034 M Na₃citrate, pH=7, re-elution of the coagulation factors with the initial PPC DEAE eluate volume of 3% NaCl+0.0034 M Na₃citrate, and 3-fold concentration by ultra-diafiltration against 0.5% NaCl +0.0085 M Na₃citrate, to obtain a concentrate with at least 40 mg protein/mL as determined by the Biuret method. All coagulation factor assays were the same as used in Examples 2 and 3.

TABLE 4

| | volume | protein | protein | FII | FII | FIX | FIX | FX | FX | PC | PC |
|---|---|---|---|---|---|---|---|---|---|---|---|
| | (ml) | [mg/ml] | [%] | [U/ml] | % | [E/ml] | % | [E/ml] | % | [E/ml] | % |
| PPC DEAE eluate | 101.0 | 15.09 | 100.0% | 25.19 | 100.0% | 28.10 | 100.0% | 25.53 | 100.0% | 25.73 | 100.0% |
| 180 min 40° C. | 102.4 | 15.07 | 101.2% | 31.75 | 127.8% | 30.07 | 108.5% | 26.56 | 105.5% | 26.80 | 105.6% |
| A/T | | | | | | | | | | | |
| re-adsorption | 491.0 | 1.45 | 46.7% | 0.01 | 0.2% | 0.04 | 0.7% | 0.01 | 0.2% | 0.07 | 1.3% |
| supernatant | | | | | | | | | | | |
| re-adsorption | 102.0 | 12.46 | 83.4% | 24.27 | 97.3% | 28.78 | 103.4% | 23.62 | 93.4% | 22.25 | 87.3% |
| eluate | | | | | | | | | | | |
| concentrate | 30.7 | 39.65 | 80.0% | 67.38 | 81.4% | 78.20 | 84.7% | 77.43 | 92.3% | 74.24 | 87.8% |

As shown in Table 4, after treatment of PPC DEAE eluate with acetyl triethyl citrate and Tween 80 and subsequent removal of these reagents, 80% of the initial coagulation factor activity was recovered.

### EXAMPLE 5

In the present example, the starting material, FEIBA DEAE-sephadex A50 eluate, was substantially the same as for the partial prothrombin complex eluate, however thrombin inhibitors were not added (U.S. Pat. Nos. 4,160,025 and 4,395,396). FEIBA Eluate was spiked with PRV stock solution in the volume ratio 1:10; P80/tributylcitrate treatment was extended to 4 h at 39° C.

TABLE 5

| | PRV/WS128.34 | | | | | |
|---|---|---|---|---|---|---|
| | 610/1 S. 152 | | | | | |
| | 1 1% w/v | | | 2 2,5% w/v | | |
| Virus | P80 + 0.15% w/v TBC | | | P80 + 0.375% w/v TBC | | |
| Experiment | 610/1 S. 159 | | | 610/1 S. 161 | | |
| Run | | calc. | | | calc. | |
| Titration | Titer | Vol. (ml) | load | Titer | Vol. (ml) | load |
| VSS | 8.69 | 30 | 10.17 | see run 1 | 30 | 10.17 |
| AUS | 7.13 | 30, 35 | 8.61 | 7.47 | 30, 86 | 8.96 |
| 10 min | 4.87 | 30, 35 | 6.35 | 4.44 | 30, 86 | 5.93 |
| 60 min | <2.11 | 30, 35 | <3.59 | <2.11 | 30, 86 | <3.60 |
| 120 min | <1.61 | 30, 35 | <3.09 | <2.11 | 30, 86 | <3.60 |
| 180 min | <1.61 | 30, 35 | <3.09 | <2.11 | 30, 86 | <3.60 |
| 240 min | <1.61 | 30, 35 | <3.09 | <2.11 | 30, 86 | <3.60 |
| Hold control | 6.79 | 30 | 8.27 | see run 1 | 30 | 8.27 |
| RF | | | >5.52 | | | >5.36 |

VSS: virus stock solution
AUS: spiked eluate

FEIBA is an auto-activated prothrombin complex based on the pro-coagulant action of both prothrombin (FII) and activated factor X (FXa) (Elsinger F, Thromb. Haemostas 1977; 38: 346 (Abstract)). The mechanism of FEIBA has been identified as the action of activated FX (FXa) in combination with prothrombin (FII). (Turecek PL, Varadi K, Gritsch H, Auer W, Pichler L, Eder G, Schwarz HP Vox Sang. 1999;77 Suppl 1:72-79).

As shown in Table 5, inactivation of PRV (one of the most detergent-resistant lipid-enveloped double-stranded DNA viruses) was achieved with Tween 80 (P80) and tributyl citrate in FEIBA DEAE eluate.

### EXAMPLE 6

FEIBA Eluate was spiked 1:10 with PRV stock suspension and subjected to P80/Acetyltriethylcitrate treatment (3 h at 39° C.).

TABLE 6

| | PRV/WS128.34 | | |
|---|---|---|---|
| Virus | 610/1 S.265 | | |
| Experiment | 1% w/v P80 + 0.36% w/v ATEC | | |
| Run | 610/1 S.268 | | |
| Titration | Titer (TCID5O/ml) | calc. Vol. (ml) | load |
| VSS | 8.43 | 30 | 9.91 |
| spiked eluate | 7.57 | 30,41 | 9.05 |
| 10 min | 6.47 | 30,41 | 7.95 |
| 60 min | 3.77 | 30,41 | 5.25 |
| 120 min | <2.11 | 30,41 | <3.59 |
| 180 min | <2.11 | 30,41 | <3.59 |
| Hold control | 7.32 | 30 | 8.80 |
| RF | | | >5.46 |

P80/ATC: 1:0,36 (w:w)

As shown in Table 6, the relatively low concentration of 1% w/v Tween 80 and 0.36% w/v acetyl triethyl citrate is able to inactivate PRV in FEIBA DEAE eluate after 120 min by exceeding the minimum of a 4log₁₀ reduction preferred for an inactivation step.

### EXAMPLE 7

The generation of FEIBA, after incubation of FEIBA DEAE eluate with 1% Tween 80 and 0.3% acetyl triethyl citrate (A/T), was tested during the production process. The process included a fivefold dilution with water after the ester/detergent treatment, the readsorption of the proteins on the same amount of DEAE sephadex A50 gel as in the DEAE sephadex adsorption step from the cryosupernatant (0.3 g/L cryoprecipitation supernatant), repeated washing (4×) of the protein loaded anion exchange gel (same as in example 4), the elution of the proteins (same as in example 4), the ultra-diafiltration of this eluate to a fifth of the original volume against a buffer of 2.5 g Na₃citrate and 5 g NaCl/L, pH=7.4, and the subsequent incubation of the obtained protein concentrate at 15° C.

The FEIBA assay was performed by mixing equal volumes of the FEIBA sample and an anti FVIII-inhibitor containing plasma (60 Bethesda Units/mL), addition of the sample volume of a phospholipid/sulfatide/kaolin reagent, incubation for 1 min at 37° C., the addition of the sample volume of a solution containing 50 mM Ca²⁺ to start the coagulation, and measuring the coagulation time in a ball coagulometer immediately after this addition.

The FEIBA unit is defined as the amount necessary to reduce the coagulation time of the anti FVIII-inhibitor plasma to 50% of the blank value.

TABLE 7

| | | | | | | | FEIBA |
|---|---|---|---|---|---|---|---|
| | Vol | protein | % total | FX | % total | FEIBA | (U/mg |
| step | (ml) | (mg/ml) | protein | (U/ml) | FX | (U/ml) | protein) |
| FEIBA DEAE eluate | 149.6 | 14.36 | 100.00% | 21.3 | 100.00% | 24 | 1.7 |
| After A/T 3 h 40° C. | 151.6 | 14.17 | 100.00% | 21.1 | 100.50% | 19 | |
| readsorption supernatant | 770.0 | 0.44 | 15.80% | 0.2 | 3.90% | | |
| readsorption eluate | 157.5 | 8.70 | 63.80% | 15.9 | 78.70% | 11 | 1.3 |
| ultra/diafiltrate | 19.8 | 63.95 | 58.90% | 120.8 | 75.20% | 47 | 0.7 |
| 10.3 h incubation | 19.8 | 63.95 | 58.90% | 116.3 | | 81 | 1.3 |
| 16.3 h incubation | 19.8 | 63.95 | 58.90% | 119.6 | | 103 | 1.6 |
| 21.0 h incubation | 19.8 | 63.95 | 58.90% | 116.6 | | 123 | 1.9 |
| 34.3 h incubation | 19.8 | 63.95 | 58.90% | 121.1 | | 144 | 2.2 |
| 41.3 h incubation | 19.8 | 63.95 | 58.90% | 120.7 | | 212 | 3.3 |

As shown in Table 7, the generation of FEIBA is unaffected by the previous Tween 80/acetyl triethyl citrate virus inactivation step.

The above results demonstrate clearly the advantage of a method according to the present invention over the known methods according to the state of the art. A composition comprising a carboxylic acid ester, in particular acetyl triethyl citrate and tributyl or triethyl citrate has a strong inactivating effect on pathogens and is however sufficiently gentle and safe in order to preserve a high level of protein function and activity to the contrary of the known combinations as for example Tween 80 with TNBP which reduces the activity of the protein. Furthermore, the composition used in the method according to the present invention can be added in smaller concentrations than the conventional pathogen inactivating compositions with however at least the same effects.

It is understood that the examples and embodiments described herein are for illustrative purposes only and that various modifications or changes in light thereof will be suggested to persons skilled in the art and are to included within the spirit and purview of this application and are considered within the scope of the appended claims. All publications, patents, and patent applications cited herein are hereby incorporated by reference in their entirety for all purposes.

What is claimed is:

1. A method for inactivating a pathogen in a protein solution which comprises adding to said protein solution either separately or together:
(a) a detergent; and
(b) an ester of a carboxylic acid formed between a carboxylic acid selected from the group consisting of monocarboxylic acids, dicarboxylic acids and tricarboxylic acids, and an alcohol which is a member selected from monohydroxy alcohols, polyhydroxy alcohols, and combinations thereof, to make a preparation, said ester being present in said preparation in a concentration of from about 0.001% to about 2% (w/w) and said detergent being present in a concentration of from about 0.001% to about 2% (w/w);
and incubating said preparation for an amount of time sufficient to inactivate said pathogen.

2. The method according to claim 1 wherein said carboxylic acid is a member selected from the group consisting of hydroxy-monocarboxylic acids, hydroxy-oligo-carboxylic acids, keto-monocarboxylic acids, keto-oligocarboxylic acids and combinations thereof.

3. The method according to claim 1 wherein said alcohol is a member selected from the group consisting of ethanol, n-butanol, dodecanol, tetradecanol, hexadecanol, octadecanol, eicosanol, glycerol, threitol, erythritol, pentitols and hexitols, pentose or hexose monosaccharide, and pentose or hexose oligosaccharide.

4. The method according to claim 1 wherein said alcohol is a short chain alcohol.

5. The method according to claim 1 wherein said carboxylic acid is a member selected from the group consisting of acetic acid, butyric acid, adipic acid, sebacic acid, succinic acid, and fumaric acid.

6. The method according to claim 1 wherein said ester is a member selected from the group consisting of mono-, di- and triglycerides of short chain fatty acids.

7. The method according to claim 6 wherein said ester is a member selected from the group consisting of monoacetyl glycerides, diacetylglycerides, triacetylglycerides, monobutyryl glycerides, dibutyrlglycerides, and tributyrylglycerides.

8. The method according to claim 2 wherein said carboxylic acid is a member selected from the group consisting of lactic acid, glycolic acid, malic acid, tartaric acid, monoacetyl tartaric acid, diacetyl tartaric acid, citric acid, isocitric acid, and gluconic acid.

9. The method according to claim 1 wherein said ester is selected from the group of citric acid esters esters consisting of triethyl citrate, tributyl citrate, and acetyl triethyl citrate.

10. The method according to claim 2 wherein said carboxylic acid is a member selected from the group consisting of pyruvic acid and oxaloacetic acid.

11. The method according to claim 1 wherein said detergent is a member selected from the group consisting of an alkali metal salt of a fatty acid, a cholic (bile) acid, a sodium- or calcium stearoyl lactyl 2-lactate, a short-chain (<C₁₄) fatty acid monoglyceride, a short-chain (<C₁₄) fatty acid diglyceride, sugar fatty acid esters, sugar glycerides, sorbitan-fatty acid esters, sorbitan-polyoxyethylene-fatty acid esters (polysorbates), and octoxynol 9 (triton X-100), nonoxynol 9 and combinations thereof.

12. The method according to claim 1, wherein said detergent is a member selected from the group consisting of a fatty acid monoglyceride which is esterified with a member selected from acetic acid, lactic acid, citric acid, tartaric acid, monoacetyl tartaric acid, diacetyl tartaric acid and combinations thereof; and a fatty acid diglyceride which is esterified with a member selected from acetic acid lactic acid, citric acid, tartaric acid, monoacetyl tartaric acid, diacetyl tartaric acid and combinations thereof.

13. The method according to claim 11 wherein said detergent is Tween 80.

14. The method according to claim 1 wherein the concentration of said ester in said preparation is from about 0.01% to about 1% (w/w).

15. The method according to claim 14 wherein the concentration of said ester in said preparation is from about 0.1% to about 0.5% (w/w).

16. The method according to claim 1 wherein the concentration of said detergent is from about 0.5% to about 1% (w/w).

17. The method according to claim 1 wherein said preparation is incubated for at least about 0.5 min.

18. The method according to claim 1 wherein said preparation is incubated at a temperature from about 0° C. to about 60° C.

19. The method according to claim 1 wherein said protein is enriched from a natural source by chromatographic or precipitation methods.

20. The method according to claim 1 wherein said protein is a member selected from the group consisting of a recombinantly produced protein and a transgenically produced protein.

21. The method according to claim 1 wherein said protein is a member selected from the group consisting of plasma, coagulation factors, immunoglobulin, albumin, antithrombin III, Cl-esterase inhibitor, α₁-antitrypsin α₁-proteinase inhibitor) and FEIBA.

22. The method according to claim 1, wherein said coagulation factor is a member selected from factor IX, and factor X.

23. The method according to claim 1 wherein said pathogen is a lipid-enveloped virus.

24. The method according to claim 23 wherein said pathogen is a member selected from the group consisting of Hepatitis virus, human immunodeficiency virus, bovine viral diarrhoea virus, herpes virus, and pseudorabies virus and combinations thereof.

25. A method for enhancing the pathogen inactivating properties of a composition comprising at least one detergent said method comprising adding to said composition a carboxylic acid ester formed between a carboxylic acid selected from the group consisting of monocarboxylic acids, dicarboxylic acids and tricarboxylic acids, and an alcohol which is a member selected from monohydroxy alcohols, polyhydroxy alcohols, and combinations thereof.

26. The method according to claim 25 wherein said ester is added to said composition in a concentration of from about 0.001% to about 20% (w/w).

27. The method according to claim 26 wherein said ester is added to said composition in a concentration of from about 0.1% to about 10% (w/w).

28. The method according to claim 27 wherein said ester is added to said composition in a concentration of from about 2% to about 5% (w/w).

29. The method according to claim 25 wherein said detergent is present in said composition in a concentration of from about 0.01% to about 20% (w/w).

30. The method according to claim 29 wherein said detergent is present in said composition in a concentration of from about 5% to about 10% (w/w).

31. The method according to claim 25 wherein said carboxylic acid is a member selected from the group consisting of hydroxy-monocarboxylic acids, hydroxy-oligo-carboxylic acids, keto-monocarboxylic acids, keto-oligocarboxylic acids and combinations thereof.

32. The method according to claim 25 wherein said alcohol is a member selected from the group consisting of ethanol, n-butanol, dodecanol, tetradecanol, hexadecanol, octadecanol, eicosanol, glycerol, threitol, erythritol, pentitols and hexitols, pentose or hexose monosaccharide, and pentose or hexose oligosaccharide.

33. The method according to claim 25 wherein said alcohol is a short chain alcohol.

34. The method according to claim 25 wherein said carboxylic acid is a member selected from the group consisting of acetic acid, butyric acid, adipic acid, sebacic acid, succinic acid, and fumaric acid.

35. The method according to claim 25 wherein said ester is a member selected from the group consisting of mono-, di- and tri-glycerides of short chain fatty acids.

36. The method according to claim 35 wherein said ester is a member selected from the group consisting of monoacetyl glycerides, diacetylglycerides, triacetylglycerides, monobutyryl glycerides, dibutyrylglycerides, and tributyrylglycerides.

37. The method according to claim 31 wherein said carboxylic acid is selected from the group consisting of lactic acid, glycolic acid, malic acid, tartaric acid, mono- and diacetyl tartaric acid, citric acid, isocitric acid, and gluconic acid.

38. The method according to claim 31 wherein said ester is selected from the group of citric acid esters consisting of triethyl citrate, tributyl citrate, and acetyl triethyl citrate.

39. The method according to claim 31 wherein said carboxylic acid is selected from the group consisting of pyruvic acid and oxaloacetic acid.

40. The method according to claim 25 wherein said detergent is a member selected from the group consisting of an alkali metal salt of a fatty acid, a cholic (bile) acid, a sodium- or calcium stearoyl lactyl 2-lactate, a short-chain (<C 14) fatty acid monoglyceride, a short-chain (<C14) fatty acid diglyceride, sugar fatty acid esters, sugar glycerides, sorbitan-fatty acid esters, sorbitan-polyoxyethylene-fatty acid esters (polysorbates), and octoxynol 9 (triton X-100), nonoxynol 9 and combinations thereof.

41. The method according to claim 25, wherein said detergent is a member selected from the group consisting of a fatty acid monoglyceride which is esterified with a member selected from acetic acid, lactic acid, citric acid, tartaric acid, monoacetyl tartaric acid, diacetyl tartaric acid and combinations thereof; and a fatty acid diglyceride which is esterified with a member selected from acetic acid lactic acid, citric acid, tartaric acid, monoacetyl tartaric acid, diacetyl tartaric acid and combinations thereof.

42. The method according to claim 40 wherein said detergent is Tween 80.
